(19) United States
(12) Patent Application Publication
(10) Pub. No.: US 2013/0189212 A1
(43) Pub. Date: Jul. 25, 2013

(54) Novel Surfactant Composition

(71) Applicants: Arun Harachandra Jawale, Dombivli (W (IN); Vaishali Amol Jumde, Navi Mumbai (IN)

(72) Inventors: Arun Harachandra Jawale, Dombivli (W (IN); Vaishali Amol Jumde, Navi Mumbai (IN)

(73) Assignee: Galaxy Surfactants Ltd., Navi Mumbai (IN)

(21) Appl. No.: 13/749,458

(22) Filed: Jan. 24, 2013

(30) Foreign Application Priority Data
Jan. 24, 2012 (IN) 3415/MUM/2011

(51) Int. Cl. A61K 8/46; A61Q 5/12; A61Q 19/10; A61Q 5/02

(52) U.S. Cl. CPC A61K 8/466; A61Q 5/02; A61Q 5/12; A61Q 19/10
USPC 424/70.19; 510/469; 510/490; 514/552

(57) ABSTRACT

A surfactant composition comprising the following elements in the indicated amounts expressed as percentage by weight: (a) 1 to 20% of at least one fatty acyl isethionate compound; (b) 0.1% to 10% of at least one acyl glycinate compound; (c) 0.1% to 20% of at least one alkyl betaine compound; and (d) 60% to 98.8% of water wherein the weight ratio of fatty acyl isethionate to acyl glycinate and alkyl betaine is in the range of 1:0.1 to 1:1 and the composition is clear, concentrated and flowable at and below 25° C. and pH at and below 6.

### CROSS REFERENCE TO RELATED APPLICATIONS

This application claims priority to 3415/MUM/2011 filed Jan. 24, 2012.

### TECHNICAL FIELD OF THE INVENTION

The present invention relates to a surfactant composition for delivery of acyl isethionate compounds. Particularly, the invention relates to a surfactant composition comprising at least one fatty acyl isethionate compound, at least one acyl glycinate compound, at least one alkyl betaine and water wherein the composition is concentrated and still remains clear, flowable and pumpable at or below pH 6 and at temperature below 25° C.

### BACKGROUND AND PRIOR ART

Acyl isethionate compounds are known to be mild surfactants with widespread use in cosmetic compositions. It provides good to excellent foam volume and quality, and generates a rich, creamy lather in personal care products. Even relatively small amounts of acyl isethionate compounds can contribute to these benefits. Acyl isethionate compounds on account of its good foamability and in particular because of its very low solubility in water, has been used for many years as a raw material in the manufacture of bar soaps.

Dissolution of Acyl isethionates compounds in an aqueous phase is desirable to extend its beneficial use into liquid personal care formulations as well. However, the solubility of a typical Acyl isethionate i.e. sodium cocoyl isethionate (SCI) in water is only about 0.01% by wt. at 25° C. and therefore it is typically available in the market place as a solid product.

For use in cosmetic compositions, the solid acyl isethionate compounds have to be dissolved under hot conditions using surfactants or solvents thus leading to high energy and time expenditure. The obligatorily hot processing also hinders the incorporation of temperature-sensitive additives, such as vitamins, dyes and the like in surfactant compositions. The processing of solid acyl isethionate compounds often results in troublesome dusting of the raw material whereby the workman is required to wear protective clothing during the manufacture or the manufacturing is required to be carried in specially sealed plants which in turn increase the cost of manufacture.

It is further desirable that these liquid personal care compositions should be both viscous and clear. This can be difficult to accomplish due to the presence of surfactants comprising higher fatty acids, salts, pH of the final formulation and relative solubility of all the components of the formulation.

In order to enhance the delivery of acyl isethionate compounds into aqueous formulations, it has been known in the art to premix one or more surfactants with acyl isethionate compounds and prepare a composition which could deliver acyl isethionate compounds at ambient temperatures.

U.S. Pat. No. 8,268,767 and U.S. Pat. No. 8,105,994 discloses personal wash cleanser composition in which large amounts of skin and hair benefiting agents such as emollients are used to reduce skin irritation. These patents do not teach anything about solubility of acyl isethionate and thus obtaining clear and flowable liquid composition.

U.S. Pat. No. 5,415,810 discloses that the dissolution of Acyl isethionate compounds in water is better achieved with the use of zwitterionic surfactants such as betaines. It states that the amount of zwitterionics used should be at least half the weight of Acyl isethionate compounds to obtain a good dissolution of the Acyl isethionate compounds. Although this patent describes mixtures comprising amphoteric surfactants in the presence of sodium cocoyl isethionate, there is no indication that these mixtures are clear and stable. Rather, these mixtures are added directly after preparation to a cosmetic product in which the isethionate compound is dissolved and stabilized by additional known additives and in which the acyl isethionate and the other surfactants of the mixture are diluted with water.

Further, two component surfactant compositions that contain Acyl isethionate compounds and an amphoacetate or a betaine are known but these systems suffer from a number of technical limitations. These two component systems are typically pastes or very viscous liquids at ambient conditions that appear to require relatively high loadings of the amphoacetates or betaines to affect the solubility of Acyl isethionate compounds. It is highly impractical to work with these viscous liquids and pastes in preparing personal care or detergent formulations. Easily flowable, two component systems have a very low total active content (less than 25%) or require a relatively high weight ratio of amphoacetate or betaine to Acyl isethionate, typically greater than one, to dissolve a high amount of Acyl isethionate compounds. For instance, in personal care formulation where the Acyl isethionate compounds to amphoacetate or betaine actives ratio needs to be equal to or greater than 1, additional Acyl isethionate compounds would have to be added and heated to high temperatures (70-90° C.) to dissolve the Acyl isethionate compounds and also the compositions are clear only at pH of above 7.5. This heating step and high pH are undesirable in the process of formulating personal care compositions.

U.S. Pat. No. 5,925,603 discloses replacing some amphoacetate content with a second anionic surfactant (SAS) or adding a second anionic surfactant to the two component surfactant compositions in order to overcome the above mentioned drawbacks.

U.S. 2009/0062406 discloses highly concentrated, transparent and colorless, aqueous concentrates comprising isethionates, taurates and cocobetaine that are flowable at room temperature. But in this system the other surfactants which are used to solubilize the isethionates, are in higher concentration than the isethionates. So the benefits of the isethionates could not be utilized upto a greater extent.

U.S. Pat. No. 8,263,538 describes a mild surfactant system comprising alkanoyl surfactants, fatty acyl isethionate and zwitterionic surfactant. However, these compositions are directly added to the personal care composition and are typically pastes or viscous liquids and hence cannot load high concentration of the surfactant system.

The key disadvantage of the prior art mixtures or the delivery systems is their poor stability at low temperature. The prior art liquid delivery systems containing acyl isethionate compounds are only soluble above 25° C. Thus, no prior art delivery systems have addressed the need of having a completely storage stable mixture or system comprising fatty acyl isethionates whereby the composition would be stable or clear at both low as well as high temperatures. It is observed that these prior art compositions tend to precipitate at or below 25° C. due to which these delivery systems become hazy. Hence it is particularly difficult to handle these systems in low temperature environments.

Also, it is observed that the surfactant compositions available in the prior art, used higher concentrations of other surfactants to solubilize the acyl isethionates. Emollient compounds are also used to solubilize the isethionate surfactants.

Hence there is a need to develop a novel surfactant composition for delivery of acyl isethionate compounds which provides improved solubilization of acyl isethionate compounds in the aqueous medium and wherein the composition exhibits good storage stability at both low and high temperatures. There is a further need to develop a surfactant system where no precipitation of Acyl isethionate compounds occurs even at temperatures below 25° C. and wherein the delivery system is suitable and easy to transport the material to the colder regions. Also there is a requirement of a concentrated surfactant system which could be loaded with high concentration of isethionate surfactants apart from other secondary surfactants and without the use of other emollient compounds.

Surprisingly, it has been found that combination of acyl glycinate compounds and alkyl betaine compounds as solubilizing system permit the solubilization of acyl isethionate in water at a considerably higher concentration

More specifically, applicants have now found that, when specific acyl isethionates are used in the specific surfactant systems noted above, in a particular ratio it can provide viscous and clear compositions. Thus, a surfactant composition comprising acyl isethionate, acyl glycinate and alkylbetaines acts as a very good composition to deliver Acyl isethionate compounds effectively in the aqueous form. Further, it has been found that surfactant compositions comprising high concentrations of acyl isethionate in combination with acyl glycinate and alkylbetaine are clear, homogeneous, flowable and pumpable at both low as well as high temperatures.

These novel surfactant compositions comprising fatty acyl isethionate, acyl glycinate and alkyl betaine are clear and flowable even at or below pH 6 which is again not taught by and even not possible in prior art compositions.

### SUMMARY OF THE INVENTION

The invention relates to a novel surfactant composition comprising: a. 1 to 20% of at least one fatty acyl isethionate compound; b. 0.1% to 10% of at least one acyl glycinate compound; c. 0.1% to 20% of at least one alkyl betaine compound; and d.60% to 98.8% of water; wherein the ratio of fatty acyl isethionate to acyl glycinate and alkyl betaine is in the range of 1:0.1 to 1:1.

According to an embodiment, the surfactant compositions of the invention are clear liquids that are pourable and pumpable even at temperatures below 25° C. The surfactant compositions of the invention can be stored for extended periods at room, elevated and cold temperatures without precipitation or decomposition of acyl isethionate compounds.

According to an embodiment, the invention relates to a personal care composition and household detergent compositions comprising the novel surfactant composition.

### BRIEF DESCRIPTION OF THE DRAWINGS

The following drawings form part of the present specification and are included to further demonstrate certain aspects of the present invention. The invention may be better understood by reference to one or more of these drawings in combination with detailed description of specific embodiments presented herein.

FIG. 1 shows the percentage increase in hair weight after a time interval of 2 hours 30 minutes as compared to initial dry weight of hair; when hair samples were treated with base formulation, formulation 1 and formulation 2.

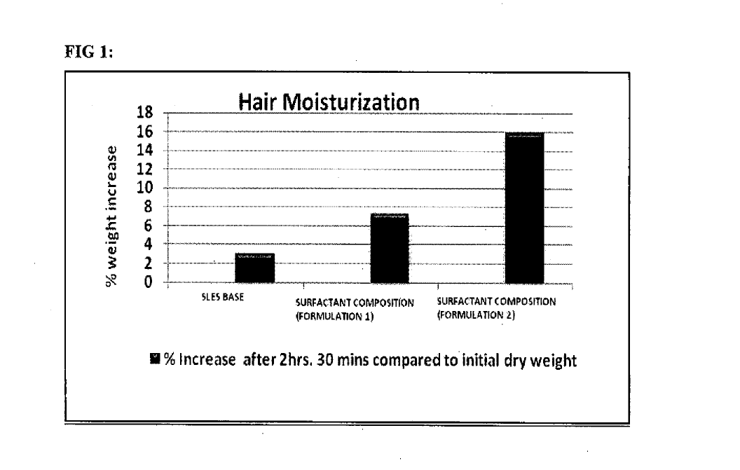

FIG. 2 shows the percentage increase in hair weight after a time interval of 2 hours when hair samples were treated with distilled water, base formulation, sodium cocoyl methyl taurate (SMCT), sodium cocoyl isethionate (SCI)-80, and sodium cocoyl glycinate (SCG)

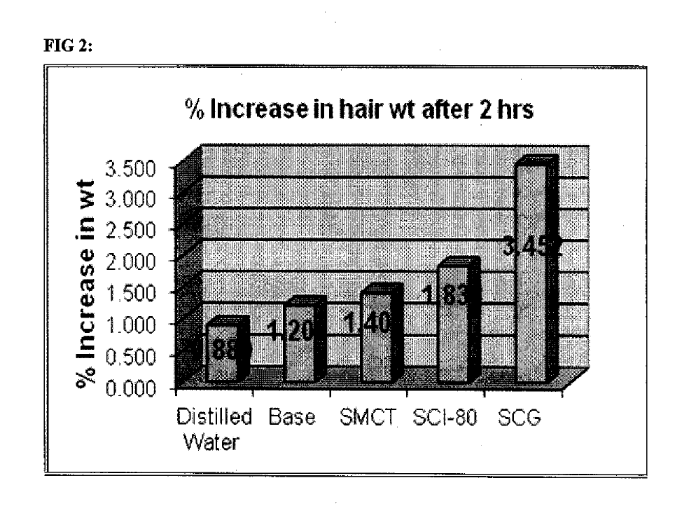

FIG. 3 shows the percentage increase in hair weight after a time interval of 2 hours and 30 minutes when hair samples were treated with distilled water, base formulation, sodium cocoyl methyl taurate (SMCT), sodium cocoyl isethionate (SCI)-80, and sodium cocoyl glycinate (SCG)

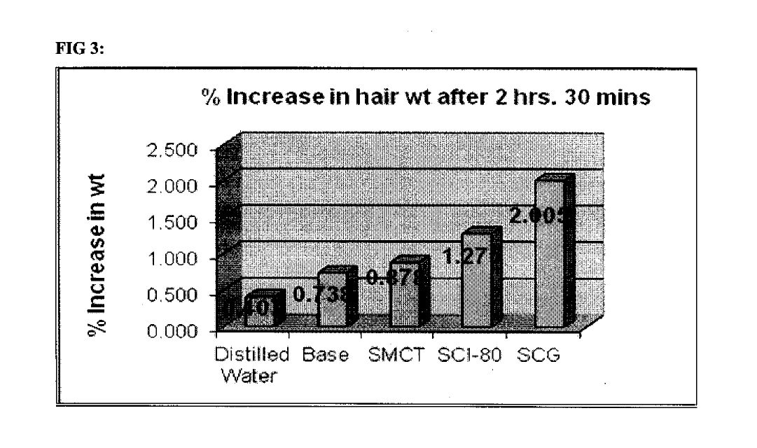

FIG. 4 shows the percentage weight loss of hair moisture at different temperatures as measured by thermo gravimetric analysis when hair samples were treated with base formulation, formulation 2, PANTENE PRO-V EXTRA MOISTURIZING SHAMPOO (P&G), FINESSE SELF ADJUSTING MOISTURIZING SHAMPOO (Lornamead International)

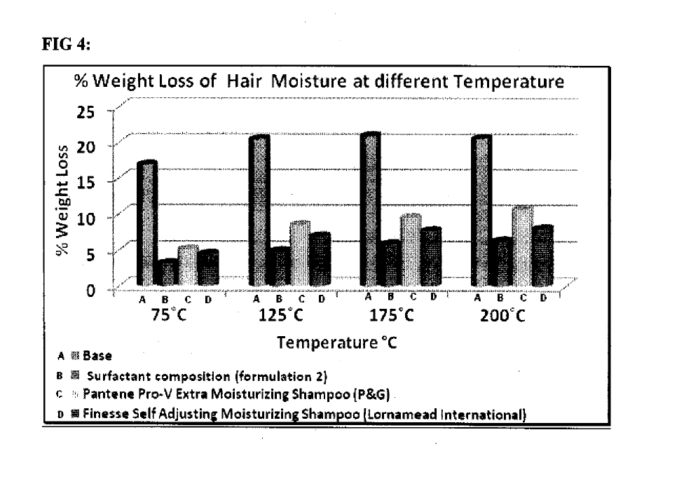

### DETAILED DESCRIPTION

In describing the embodiments of the invention, specific terminology is resorted for the sake of clarity. However, it is not intended that the invention be limited to the specific terms so selected and it is to be understood that each specific term includes all technical equivalents that operate in a similar manner to accomplish a similar purpose.

The present invention relates to a surfactant composition for delivery of acyl isethionate compounds in an aqueous form such that the composition is clear, concentrated and flowable even at acidic pH.

The surfactant composition comprises 1 to 20% of at least one fatty acyl isethionate compound, 0.1% to 10% of at least one acyl glycinate compound, 0.1 to 20% of at least one alkylbetaine compound and 60% to 98.8% of water to deliver acyl isethionate compounds in an aqueous form. The surfactant composition remains clear, flowable and pumpable at low and high temperature storage conditions.

According to another embodiment, the surfactant composition comprises 1 to 18% of at least one fatty acyl isethionate, 1 to 8% of at least one acyl glycinate compound, 1 to 17% of at least one alkylbetaine and 64% to 97% of water to deliver acyl isethionate compounds in an aqueous form.

According to another embodiment, the surfactant composition comprises 10% of at least one fatty acyl isethionate, 2% of at least one acyl glycinate compound, 7% of at least one alkylbetaine and 81% of water to deliver acyl isethionate compounds in an aqueous form.

According to yet another embodiment, the surfactant composition comprises 15% of at least one fatty acyl isethionate, 5% of at least one acyl glycinate compound, 10% of at least one alkylbetaine and 70% of water to deliver acyl isethionate in an aqueous form.

According to an embodiment, the fatty acyl isethionate compound is selected from the compounds of formula: R—CO₂—(CH₂)ₙ—SO₃⁻M⁺, wherein, R is an alkyl or alkenyl group of 6 to 26 carbon atoms, n is an integer from 2 to 4, preferably 2, and m is an alkali or alkaline earth metal such as sodium, potassium, lithium, magnesium, ammonium or substituted ammonium.

According to another embodiment, the fatty acyl isethionate compound has less than 22% of the total fatty acyl isethionate compounds having fatty acyl group with alkyl chain length of C16 or greater. The inventors have surprisingly observed that the fatty acyl isethionates with these chain lengths enable to obtain clear and flowable surfactant composition.

According to another embodiment, the fatty acyl isethionate compound also contains free fatty acid and/or fatty acid salt as by products.

According to yet another embodiment, the fatty acyl isethionate compound of the invention comprises less than 42% of free fatty acid and fatty acid salt groups having C16 or greater chain length of fatty acyl group.

According to yet another embodiment, the fatty acyl isethionate compound of the invention is sodium cocoyl isethionate (SCI). SCI are considered exceptionally mild for the skin, hair, and eyes, and are particularly tolerant to hard water. They leave behind a great skin feel and impart good foamability and generate creamy and stable lather. SCI is good for all skin or hair type. Example of SCI used in the delivery system of the invention is GalSoft SCI 80 (manufactured by Galaxy Surfactants)

According to an embodiment, the acyl glycinate compound is selected from the compounds of the formula: R—CO—NH—CH₂—COOX wherein, R is a straight or branched chain alkyl or alkenyl residue having from 2 to 18 preferably 8 to 18 carbon atoms, X is hydrogen, an alkali or alkaline earth metal, the ammonium radical, a primary, secondary or tertiary amine group or a hydroxylalkylamine group,

According to an embodiment, the acyl glycinate compound is sodium cocoyl glycinate (SCG). SCG is a mild co-surfactant which gives rich creamy foam in personal care compositions. It provides excellent foam in combination with surfactants such as Cocamidopropyl Betaine or Coco Betaine, as comparable to standard sodium lauryl ether sulfate (SLES)/Betaine systems. SCG based formulations require lesser amounts of viscosity modifiers compared to other amino acid surfactants like acylglutamates or sarcosinates. SCG also provides excellent rinsability over conventional co-surfactants like cocamidopropylbetaines, taurate surfactants, acylglutamates and monoalkylphosphates. Example of SCG used in the delivery system of the invention is GalSoft SCG (manufactured by Galaxy Surfactants). Other acyl glycinates such as C₁₀-C₂₄ acyl glycinates are also known to be skin friendly surfactants. These materials have a desirable skin feel and are mild and do not cause any perceptible irritation.

According to an embodiment, the alkyl betaine is selected from the compounds of the formula: R—N⁺—(CH₃)₂—Z⁻X⁺; wherein R is a straight or branched chain alkyl or alkenyl residue having from 2 to 18 preferably 8 to 18 carbon atoms, Z is selected from the group consisting of CO₂, SO₃, and PO₂; and X is hydrogen, an alkali or alkaline earth metal, the ammonium radical, a primary, secondary or tertiary amine group or a hydroxylalkylamine group.

According to an embodiment, the alkyl betaine compound is Cocobetaine. Cocobetaine is used as a foam booster in cleansing systems. It is a medium strength surfactant used in bath products like hand soaps & shampoos. It is also used in cosmetics as an emulsifying agent and thickener to reduce irritation caused by purely ionic surfactants. It also serves as an antistatic agent in hair conditioners and has antiseptic properties, making it suitable for personal sanitary products. Example of Cocobetaine compounds used in the delivery system of the invention is Galaxy CB (manufactured by Galaxy Surfactants)

According to yet another embodiment of the invention, the amount of acyl glycinate and alkylbetaine as solubilizing system is equal to or less than fatty acyl isethionate. Thus, the active weight ratio of acyl isethionate to acyl glycinate and alkylbetaine is in the range of 1:0.1 to 1:1, preferably from 1:0.5 to 1:1, most preferably from 1:0.9 to 1:1.

According to yet another embodiment the active weight ratio of alkylbetaine to acyl glycinate is in the range of 1:0.1 to 1:1. Thus, the amount of alkylbetaine will always be higher than that of acyl glycinate in the surfactant composition.

It is important to note that, in any cleansing composition, anionic surfactants should be more than other surfactants e.g. cationic, amphoteric, zwitterionic, nonionic, etc. Anionic surfactant will give a better cleansing property to the composition. Therefore, although it is also possible to make clear and flowable composition using higher ratio i.e. using high amount of alkyl betaine it is not advisable to add more amphoteric surfactants e.g. alkyl betaine. Also adding more of acyl glycinate surfactant does not give clear solution.

According to yet another embodiment the active weight ratio of acyl isethionate to acyl glycinate and alkylbetaine is 1:1.

The surfactant compositions of the present invention are new, useful and unobvious to those skilled in the art of making stable delivery system containing SCI, which has very limited water solubility at 25° C.

The inventive system is a low viscous liquid, is easy to handle and can be processed even in cold conditions. The viscosity of the surfactant composition ranges between 6000-25000 cps. Thus, it is viscous enough to give the final formulation, the desired viscosity without using any polymeric viscosity enhancers. Personal care products such as shampoos should have a particular viscosity so that the liquid solution does not flow into the eyes but stays on the hair and also the dispensing of the liquid shampoo becomes comfortable.

According to an embodiment surfactant compositions of the invention are clear liquids that are pourable and pumpable even at temperatures below 25° C. The surfactant compositions of the invention can be stored for extended periods at room, elevated and cold temperatures without precipitation or decomposition of acyl isethionate compounds.

According to another embodiment, the surfactant compositions are clear and stable at and below pH 6.

According to another embodiment, the surfactant compositions are clear and stable typically in the pH range of 4-6 which is also called skin pH as the pH of our skin is acidic. Hence compositions with acidic pH are more suitable than compositions with alkaline pH such as soaps which are harsher.

According to yet another embodiment, the surfactant composition can further optionally include minor amounts of other anionic or nonionic or zwitterionic surfactants that are believed not to be detrimental to the invention. The anionic or nonionic or zwitterionic surfactants can be added to further enhance the pourability, stability and/or performance of surfactant composition.

According to an embodiment, the anionic surfactants that can be further added include one or more of acyl taurates, alkyl ether sulfates, alkyl or alkenyl sulfates, alkyl glyceryl ether sulfates, sulphosuccinates, sulfosuccinamates, acyl sarcosinates, fatty acyl amino acids other than acyl glycinates and sarcosinates, sulphoacetates, monoalkyl phosphate esters, di-alkyl phosphate esters, mono-alkyl ether phosphate esters, di-alkyl ether phosphate esters, alpha-olefin sulfonates, acyl lactates, alkyl ether carboxylates and glyceryl ether carboxylates. However, those skilled in the art will appreciate that it is possible to utilize other anionic surfactants known in the art without departing from the scope of the invention.

According to an embodiment these anionic surfactants can be present in an amount from about 0.1% to 20%.

According to an embodiment, the nonionic surfactants that can be further added include one or more of alkanolamides, ethylene glycol monostearate, ethylene glycol distearate and alkyl ethoxylated alcohols and alkyl ethoxylated/propoxylated alcohols. When adding some of these nonionics, care must be taken not to diminish the clarity, foaming and lathering properties of the surfactant compositions. However, those skilled in the art will appreciate that it is possible to utilize other nonionic surfactants known in the art without departing from the scope of the invention.

According to an embodiment these nonionic surfactants can be present in an amount from about 0.1% to 20%.

According to an embodiment the zwitterionic surfactants include one or more of simple betaines, amidobetaines, sulphobetaines, alkyl iminoacetates, alkyliminodiacetates, alkyl iminopropionates, alkyl iminodipropionate, and alkyl amine oxides. The Zwitterionics surfactants are added to provide primarily foam boosting and/or conditioning properties to the delivery system. However, those skilled in the art will appreciate that it is possible to utilize other zwitterionic surfactants known in the art without departing from the scope of the invention.

According to an embodiment these zwitterionic surfactants can be present in an amount from about 0.1% to 20%.

According to still further embodiment very small amounts of other chemical may be added to the surfactant composition of this invention. These additives include but are not necessarily limited to pH adjusting chemicals such as acids, bases and buffers, e.g. sodium hydroxide, citric acid, triethanolamine, salicylic acid; lower molecules weight alcohols containing more than one hydroxyl group, e.g. ethylene glycol, propylene glycol, diethylene glycol, triethylene glycol, glycerin; antioxidants, e.g. BHT; preservatives, e.g. methyl and propyl parabens and the like; inorganic salts, e.g. alkali and alkali metal halides, acetates, carboxylates, sulfonates, and citrates; thickening and conditioning agents such as guar and cellulosics and chemical derivatives of guar and cellulosic.

According to yet another embodiment, the invention further relates to the process of preparation of the surfactant composition.

According to an embodiment the process of preparation of surfactant composition involves stirring the mixture of acyl glycinate, alkylbetaine and water at an elevated temperature. The process further involves adding the acyl isethionate compound slowly with continuous stirring till complete solubilization of acyl isethionates is achieved. The solution is then cooled to room temperature. Finally the pH and viscosity of the solution is measured and pH is adjusted with pH adjusting agents if required.

According to an embodiment, the invention relates to personal care composition and household detergent products comprising the surfactant composition comprising: a. 1 to 20% of at least one fatty acyl isethionate compound; b. 0.1% to 10% of at least one acyl glycinate compound; c. 0.1% to 20% of at least one alkyl betaine compound; and d. 60% to 98.8% of water.

According to an embodiment, the surfactant composition is used in personal care compositions such as shampoos, hand soaps, body washes, face washes, hand washes, shower gels, baby bubble bath, antiseptic washes and the like. Hence, formulators of personal care products and other household detergent products will find many advantages in using this inventive surfactant composition.

This inventive surfactant composition can be mixed with other components of personal care formulation at low temperature i.e. at cold conditions as well, thereby reducing and/or eliminating high energy heating processes which are undesirable in such compositions.

Due to achievement of high concentration of SCI in the surfactant composition, batch manufacturing time is reduced thus increasing the plant output. It further results in overall reduction in energy and manpower costs. It also results in reduction in handling costs due to consolidated warehousing. Also since the delivery system has high concentration of the surfactants, bulk storage is decreased. Further, quality control time is reduced with the elimination of many individual surfactants to purchase and monitor, and many unwanted other material can be avoided in the final formulation;

In addition for SCI based formulations, the inconvenience of adding solid SCI flakes can be avoided thus making the process devoid of dust particles which is a potential health hazard.

The following examples will serve to illustrate the invention, but should not be construed to limit the invention. All parts, percentages, ratios and the like in these examples and in the remainder of the specification and claims are in weight/weight percentages of the total composition unless otherwise indicated.

### EXAMPLES

In order to demonstrate the improved flowability and stability with respect to appearance and viscosity, applicant compared prior art compositions with the surfactant composition prepared according to embodiments of the invention, the results of which are as set forth in Table 1 and 2 below.

TABLE 1
Flowability and stability data for the composition prepared as per the embodiments of the present invention

| | As per the embodiments of the present invention | | | | | | | | |
|---|---|---|---|---|---|---|---|---|---|
| | % (Active Basis) | | | | | | | | |
| Composition | Example 1 | Example 2 | Example 3 | Example 4 | Example 5 | Example 6 | Example 7 | Example 8 | Example 9 |
| Sodium | 10 | 10 | 10 | 15 | 1 | 17 | 1 | 17 | 20 |
| cocoyl | | | | | | | | | |
| isethionate | | | | | | | | | |
| Sodium | 2 | 2 | 5 | 5 | 0.5 | 2 | 0.5 | 2 | 10 |
| cocoyl | | | | | | | | | |
| glycinate | | | | | | | | | |
| Cocobetaine | 8 | 7 | 5 | 10 | 0.5 | 15 | 0.5 | 15 | 10 |
| Free coco | 1.5 | 1.5 | 1.5 | 2.3 | 0.15 | 2.5 | 0.15 | 2.5 | 3.0 |
| fatty acid* | | | | | | | | | |
| Sodium | 1.8 | 1.6 | 1.2 | 2.4 | 0.12 | 3.45 | 0.12 | 3.45 | 2.4 |
| chloride** | | | | | | | | | |
| Water | q.s. to 100 | q.s. to 100 | q.s. to 100 | q.s. to 100 | q.s. to 100 | q.s. to 100 | q.s. to 100 | q.s. to 100 | q.s. to 100 |
| pH as is at 25° C. | 5.5 | 5.5 | 6.0 | 6.0 | 5.5 | 6.0 | 5.5 | 6.0 | 6.0 |
| Viscosity | 19496 | 21058 | 6905 | 10498 | 700 | 25000 | 700 | 25000 | 30000 |
| (with spindle | | | | | | | | | |
| S 96, 30 rpm, | | | | | | | | | |
| T-bar | | | | | | | | | |
| spindle, | | | | | | | | | |
| Temp. 22 | | | | | | | | | |
| deg. C., Unit - | | | | | | | | | |
| cps) | | | | | | | | | |
| Appearance | Clear & | Clear & | Clear & | Clear & | Clear & | Clear & | Clear & | Clear & | Clear & |
| of LDS | Flowable | Flowable | Flowable | Flowable | Flowable | Flowable | Flowable | Flowable | Flowable |
| stored for 24 | liquid | liquid | liquid | liquid | liquid | liquid | liquid | liquid | liquid |
| hours | @20° C. | @22° C. | @25° C. | @25° C. | @22° C. | @25° C. | @22° C. | @25° C. | @25° C. |

*free coco fatty acid is impurity contained in sodium cocoyl isethionate
**sodium chloride is impurity contained in sodium cocoyl glycinate/taurate/sarcosinates/and betaines

TABLE 2
Comparative flowability and stability data with respect to compositions known in the art.

| | Comparative example % Active Basis) | | | | | | | | |
|---|---|---|---|---|---|---|---|---|---|
| | Example | Example | Example | Example | Example | Example | Example | Example | Example |
| | 10 | 11 | 13 | 14 | 15 | 16 | 17 | 18 | 19 |
| Sodium cocoyl isethionate | 10 | 10 | 10 | 10 | 10 | 10 | 5 | 50 | 60 |
| Sodium cocoyl glycinate | — | 2 | 2 | — | | 5 | | 30 | 30 |
| Cocobetaine | 10 | — | — | 5 | 8 | — | 20 | 20 | 30 |
| Cocoamidopropyl betaine | — | 8 | — | — | — | — | — | — | — |
| Cocoamidopropyl sulfo | — | — | 8 | — | — | — | — | — | — |
| betaine | | | | | | | | | |
| C8-10 alkyl amido betaine | — | — | — | — | — | 5 | — | — | — |
| Cocoamphoacetate | — | — | — | — | — | — | — | — | — |
| Sodium cocoyl methyl | — | — | — | — | 2 | — | 5 | — | — |
| taurate | | | | | | | | | |
| Sodium Lauroyl | — | — | — | 5 | — | — | — | — | — |
| Sarcosinate | | | | | | | | | |
| Free coco fatty acid* | 2.5 | 2.5 | 2.5 | 2.5 | 2.5 | 2.5 | 2.5 | 12.5 | 15 |
| Sodium chloride** | 1.45 | 2.2 | 2.2 | 1.19 | 2.1 | 2.3 | 2.5 | 6.3 | 7 |
| Water | q.s. to 100 | q.s. to 100 | q.s. to 100 | q.s. to 100 | q.s. to 100 | q.s. to 100 | q.s. to 100 | q.s. to 100 | q.s. to 100 |
| pH | 7.0 | 7.0 | 6.9 | 6.9 | 7.0 | 6.9 | 7.0 | 7.0 | 7.0 |
| Viscosity (spindle S 96, | 2218 | Thick, | 1437 | 625 | 26932 | 2500 | 20000 | Thick | Thick |
| 30 rpm, T-bar spindle, | | gelly. | | | (S 96 | | | paste | paste |
| Temp. 22 deg. C., Unit - | | | | | spindle @ | | | | |
| cps) | | | | | 12 rpm) | | | | |
| Appearance of LDS stored | Hazy | Hazy | Hazy | Hazy | opaque | Hazy | Clear | Opaque | Opaque |
| @25° C. for 24 hours | liquid | Thick | liquid | liquid | thick | liquid | liquid | thick | thick |
| | | gelly | | | paste | | | paste | paste |

It can be observed from Table 1 that a blend of surfactants according to embodiments of the invention having a 1:1 ratio of acyl isethionate to acyl glycinate and betaine shows that it requires an equal or less amount of solubilising surfactant system (acyl glycinate and alkylbetaine) as compared to prior art compositions where large quantity of solubilizing surfactant systems are required.

From the Table 1, it was observed that the surfactant composition according to embodiments of the present invention was found to be clear and stable during extended storage and no precipitation of sodium cocoyl isethionate was observed. Thus Table 2 shows that the surfactant composition according to the embodiments of the invention overcomes the limitations especially of low temperature storage stability as compared to prior art compositions.

From the comparative examples as shown in Table 2, it is clearly observed that the compositions known in the art do not give clear, flowable liquid mass if we maintain the ratio of acyl isethionate to other surfactants to 1:1.whereas, this can be achieved by the inventive surfactant combinations shown in examples 1 to 9 in Table 1.

Also it was observed from Table 2 that the surfactant system comprising isethionate as are known in the art are hazy even at neutral pH whereas the surfactant composition according to embodiments of the invention comprising fatty acyl isethionate, acyl glycinate and alkyl betaine showed clear solution even at pH lower than 6.0 i.e. acidic pH.

Further it can be observed from Table 2, Example 10 that even when the sodium cocoyl Isethionate and coco Betaine is in a 1:1 ratio, a clear liquid is not obtained whereas as shown in Table 1- examples 1 to 9, the surfactant compositions as per the embodiments of the invention comprising both acyl glycinate and coco betaine, provides a clear liquid and that too in 1:1 ratio with acyl isethionate.

Also it can be observed from Table 2 that acyl sarcosinate (e.g. sodium lauroyl sarcosinate) and acyl taurate (sodium cocoyl methyl taurate) which have been used in the prior art compositions instead of acyl glycinate do not show good results in 1:1 ratio.

Another aspect of the present invention is the use of the novel surfactant composition in skin and hair care formulations.

Sample/Formulation preparation to determine moisture retention of hair:

Following is the example of personal care products prepared by incorporating the inventive surfactant compositions into the base formulations.

Base Formulation

| Sr. No. | Ingredients | % w/w |
|---|---|---|
| 1 | sodium lauryl | 25.00 |
| | ether sulfate | |
| 2 | Cocoamidopropyl | 6.00 |
| | betaine | |
| 3 | cocomono | 2.00 |
| | ethanolamide | |
| 4 | Preservative | 0.70 |
| 5 | EDTA | 0.10 |
| 6 | Distilled Water | q.s. to 100 |

Formulation 1:

| Sr. No. | Ingredients | % w/w |
|---|---|---|
| 1 | Base Formulation | 99 |
| 2 | Surfactant | 1 |
| | composition of | |
| | example 1 | |

Formulation 2:

| Sr. No. | Ingredients | % w/w |
|---|---|---|
| 1 | Base Formulation | 95 |
| 2 | Surfactant | 5 |
| | composition of | |
| | example 1 | |

Hair moisturization is one of the most important desired properties of hair cleansing formulations, due to which hair looks healthy, manageable and better in sensory feel. The amount of moisture retention was determined by measuring changes in weight by the following two methods.

Method 1:

To determine moisture retention of hair, an in-house method was followed which is as mentioned below:
1. 200 ml of 10% solution of formulation 1 and the base (formulation-2) were prepared.
2. 200 ml distilled water was taken in another beaker (blank).
3. Dried and weighed hair swatches (Indian virgin hair) were labeled with respective sample names and taken for further study.
4. Hair swatches are dipped into the solutions and held with clips. The hair swatches were kept for 1 hour in the same condition.
5. After 1 hour all the swatches were removed and the excess solution or water was dripped out by means of filter paper (Do not wipe out excessively or roughly as hair loss may occur).
6. The swatches were then dried at temperature of around 25-27° C. and RH 55-60%.
7. The swatches were weighed after 2 hrs 30 mins and the moisture retention percentage of each sample was calculated from the difference between the initial weights and weight after 2 hrs 30 mins.

Hair swatches can be kept at the above condition for some more time and changes in the weight can be recorded after regular intervals to understand the moisture retention pattern of the samples over a prolonged period.

It was observed that the formulations containing the surfactant compositions (Formulations 1 and 2) provide better moisturization compared to the base formulation.

Moisture retention study on individual surfactants were also performed to establish the better moisturizing benefit of SCG over the other surfactants. Surfactants such as sodium cocoyl methyl taurate (SMCT), SCI, and SCG) were used on 1% active basis in base formulation for individual comparison in terms of retention of hair Moisturization.

Measuring % Increase in Moisture:

% increase in moistures using all individual surfactants was measured after 2 hours and again after 2 hour 30 mins as given below. Calculation: % Increase in Moisture=[(Weight of hair swatch after 2 hrs. 30 min−Initial weight of hair swatch)/Initial weight of hair]*100

After compilation of all results/observations it was found that SCG retains more % of moisture compared to other mild surfactants-SMCT, SCI, SLES+CAPB(Base formulation). From this observation it was decided to select SCG +SCI combination for further study of concentrate development.

Method 2:

The % weight loss of hair moisture is determined by Thermo gravimetric analysis (TGA). This analysis was performed at SAS laboratories, Ghatkopar, Mumbai.

% Weight loss at various temperatures

| Sample | 75° C. | 125° C. | 175° C. | 200° C. |
|---|---|---|---|---|
| Base | 16.92 | 20.51 | 21.02 | 20.61 |
| Formulation 2 | 3.19 | 4.90 | 5.88 | 6.32 |
| Pantene Pro-V Extra Moisturizing | 5.11 | 8.51 | 9.49 | 10.65 |
| Shampoo (P&G) | | | | |
| Finesse Self Adjusting | 4.48 | 6.96 | 7.71 | 8.02 |
| Moisturizing Shampoo (Lornamead | | | | |
| International) | | | | |

Pantene Pro-V Extra Moisturizing Shampoo (P&G) includes, water, sodium laureth sulfate, sodium lauryl sulfate, sodium chloride, glycol distearate, dimethicone, fragrance, cocamidopropyl betaine, sodium citrate, cocamide MEA, sodium xylenesulfonate, citric acid, guar hydroxypropyltrimonium chloride, sodium benzoate, ammonium laureth sulfate, tetrasodium EDTA, polyquaternium-76, panthenyl ethyl ether, panthenol, methylchloroisothiazolinone, methylisothiazolinone

Finesse Self Adjusting Moisturizing Shampoo (Lornamead International) includes, water (aqua), ammonium lauryl sulfate, ammonium laureth sulfate, dimethiconol, cocamide MEA, glycerin, silk powder (serica), hydrolyzed soy protein, cocamidopropyl betaine, fragrance (parfum), PEG-5 cocamide, carbomer, amodimethicone, guar hydroxypropyltrimonium chloride, ammonium chloride, TEA-dodecylbenzenesulfonate, tetrasodium EDTA, DMDM hydantoin, C11-15 pareth-7, laureth-9, PEG-45M, trideceth-12, PPG-9, methylchloroisothiazolinone, methylisothiazolinone, mica (CI 77019), titanium dioxide (CI 77891).Lesser the % Weight Loss, More the % Moisture Retention

Method/Protocol for TGA: The TGA measurements were conducted using the Perkin Elmer Instrument for moisture determination in hair samples.Conditions: Measuring Cell—Perkin Elmer; Weighing PAN—Alumina 40 μL Sample Preparation—As such treated and dried hair samples (10 to 11 mg) were exposed to heat and continuous measurements were recorded automatically. All measurements were recorded while heating the samples from 30 to 300° C. at the rate of 10° C. per minute. The moisture content is measured at every 10 deg C. Atmosphere—Nitrogen 20 Cm3/minute. The calibration of the instrument was done using Indium as a standard. Finally the % weight loss was determined by the below mentioned formula: % weight loss of moisture=weight-final weight (at specific Temp)}/Initial weight*100

From the figures it is evident that the formulation comprising the inventive surfactant composition prevents the loss of moisture from skin and hair and delivers the improved moisturizing benefits as compared to the market sample 1—Pantene Pro-V, extra moisturizing shampoo (P & G) and market sample 2—Finesse Moisturizing shampoo (This is an international brand in India Imported & marketed by Lornamead Corporate Services). Following are the examples of personal care products prepared by incorporating the inventive surfactant compositions.

Formulation 3: Mild Baby Bath

| Ingredients | % w/w |
|---|---|
| Inventive surfactant composition | 50.00 |
| Glycerin | 2.00 |
| Kathon CG | 0.10 |
| EDTA disodium | 0.05 |
| Aculyne-60 | 0.10 |
| Distilledwater | q.s. upto 100 |
| Appearance | Clear |
| pH as such | 5.5 |

Formulation 4:

Formulation-
Skin Smoothening Face Wash

| Ingredients | % w/w |
|---|---|
| Inventive Surfactant | 40.0 |
| composition | |
| Sodium lauroyl sarcosinate | 10.0 |
| PEG-7 GC | 2.0 |
| Glycerin | 5.0 |
| EDTA disodium | 0.05 |
| Preservative blend | 0.5 |
| Distilled Water | q.s. upto 100 |
| pH | 5.7 |

Formulation 5:

Formulation-Refreshing Body Wash

| Ingredients | % w/w |
|---|---|
| Inventive surfactant composition | 60.0 |
| Sodium lauroyl sarcosinate | 10.0 |
| PEG-7 GC | 2.0 |
| Glycerin | 5.0 |
| Aculyne-60 | 0.10 |
| Tocopherol acetate | 0.5 |
| Rice protein | 0.5 |
| EDTA disodium | 0.05 |
| Preservative blend | 0.5 |
| Distilled Water | q.s. upto 100 |
| pH | 5.5 to 6.5 |

Surprisingly, it was noticed that incorporating the inventive surfactant composition in hair care compositions results in improved moisturizing effect. This improvement in moisturizing effect is not possible by using prior art surfactant compositions.

This invention may be embodied in other forms or carried out in other ways without departing from the spirit or essential characteristics thereof. The embodiments given hereinbefore are therefore to be considered as in all respects illustrative and not restrictive, the scope of the invention being indicated by the appended claims, and all changes which come within the meaning and range of equivalency are intended to be embraced therein. It is to be understood that no limitation with respect to the specific embodiments illustrated is intended or should be inferred.

We claim:

1. A surfactant composition comprising:
a. 1 to 20% of at least one fatty acyl isethionate compound;
b. 0.1% to 10% of at least one acyl glycinate compound;
c. 0.1% to 20% of at least one alkyl betaine compound; and
d. 60% to 98.8% of water.
wherein the ratio of fatty acyl isethionate to acyl glycinate and alkyl betaine is in the range of 1:0.1 to 1:1. ,

2. The surfactant composition according to claim 1, wherein fatty acyl isethionate compound is selected from the compounds of formula:
R—CO₂—(CH₂)ₙ—SO₃⁻M⁺
wherein,
R is an alkyl or alkenyl group of 6 to 26 carbon atoms,
wherein preferably less than 25% of the total fatty acyl isethionate compound consist of fatty acyl group having alkyl chain length of C₁₆ or greater; and less than 50% of chain length of free fatty acid and fatty acid salt groups combined are of chain length C₁₆ or greater
n is an integer from 2 to 4, preferably 2, and
m is an alkali or alkaline earth metal such as sodium, potassium, lithium, magnesium, ammonium or substituted ammonium.

3. The surfactant composition according to claim 1, wherein acyl glycinate compound is selected from the compounds of formula:
R—CO—NH—CH₂—COOX
wherein,
R is a straight or branched chain alkyl or alkenyl residue having from 2 to 18 preferably 8 to 18 carbon atoms,
X is hydrogen, an alkali or alkaline earth metal, the ammonium radical, a primary, secondary or tertiary amine group or a hydroxylalkylamine group.

4. The surfactant composition according to claim 1, wherein alkyl betaine compound is selected from the compounds of formula:
R—N⁺—(CH₃)₂—Z⁻X⁺
Wherein R is a straight or branched chain alkyl or alkenyl residue having from 2 to 18 preferably 8 to18 carbon atoms,
Z is selected from the group consisting of CO₂, SO₃, and PO₂; and
X is hydrogen, an alkali or alkaline earth metal, the ammonium radical, a primary, secondary or tertiary amine group or a hydroxylalkylamine group.

5. The surfactant composition of claim 1, wherein the ratio of alkylbetaine compound to acyl glycinate compound is in a range of 1:0.1 to 1:1.

6. The surfactant composition according to claim 1 comprising:
a. 10% of at least one fatty acyl isethionate compound;
b. 2% of at least one acyl glycinate compound;
c. 7% of at least one alkyl betaine compound; and,
d. 81% of water.

7. The surfactant composition according to claim 1 comprising:
a. 15% of at least one fatty acyl isethionate compound;
b. 5% of at least one acyl glycinate compound;
c. 10% of at least one alkyl betaine compound; and,
d. 70% of water.

8. The surfactant composition according to claim 1, wherein the fatty acyl isethionate compound is sodium cocoyl isethionate.

9. The surfactant composition according to claim 1, wherein the acyl glycinate compound is sodium cocoyl glycinate.

10. The surfactant composition according to claim 1, wherein the alkyl betaine compound is cocobetaine.

11. The surfactant composition according to claim 1, further comprising one or more of anionic, non-ionic or zwitterionic surfactants.

12. A personal care composition comprising the surfactant composition of claim 1.

13. A household detergent composition comprising the surfactant composition of claim 1.
